(19) United States
(12) Patent Application Publication
(10) Pub. No.: US 2026/0092095 A1
(43) Pub. Date: Apr. 2, 2026

(54) INTERLEUKIN-2 FOR USE IN TREATING AUTISM SPECTRUM DISORDER

(71) Applicants: INSERM (INSTITUT NATIONAL DE LA SANTÉ ET LA RECHERCHE MÉDICALE), PARIS (FR); SORBONNE UNIVERSITE, PARIS (FR); ASSISTANCE PUBLIQUE-HÔPITAUX DE PARIS, PARIS (FR)

(72) Inventors: David Klaatzman, Paris (FR); Pierre Ellul, Paris (FR); Michelle Rosenzwajg, Paris (FR); Richard Delorme, Paris (FR); Gwladys Fourcade, Paris (FR)

(73) Assignees: INSERM (INSTITUT NATIONAL DE LA SANTÉ ET LA RECHERCHE MÉDICALE), Paris (FR); SORBONNE UNIVERSITE, Paris (FR); ASSISTANCE PUBLIQUE-HÔPITAUX DE PARIS, Paris (FR)

(21) Appl. No.: 19/110,627

(22) PCT Filed: Sep. 12, 2022
(86) PCT No.: PCT/EP22/75319

§ 371 Date: Mar. 11, 2025

(51) Int. Cl. C07K 14/55; A61K 38/00

(52) U.S. Cl. CPC C07K 14/55; A61K 38/00

(57) ABSTRACT

The invention relates to the use of IL-2 in treating autism spectrum disorder (ASD) in a subject, as well as in preventing ASD in children, by administering the IL-2 to a child at risk of ASD or to a pregnant mother at risk of having a child with ASD.

The present invention relates to prevention and treatment of autism spectrum disorder.

### BACKGROUND OF THE INVENTION

Autism spectrum disorders (ASD) are a group of neurodevelopmental conditions characterized by deficits in social communication and interaction along with restricted, repetitive, behaviors. In addition to genetic risk factors, the immune system is an important factor in the emergence of ASD. Epidemiological studies have highlighted an increased risk of ASD in children of mothers who had infections or autoimmune disorders during pregnancy. Mechanistically, animal studies have shown that the links between maternal immune activation (MIA) and ASD are underpinned by secretion of IL-17α, by maternal Th17 lymphocytes, that crosses the placental barrier and binds to IL-17α receptors on fetal neurons disrupting neurodevelopment.

Although there are psychopharmacological options available for the treatment of unspecific symptoms associated with ASD such as irritability and hyperactivity, to date, no medications have consistently been shown to reliably improve core symptoms of ASD.

Furthermore, there are no effective options available to prevent occurrence and development of ASD.

### SUMMARY OF THE INVENTION

The inventors now propose to use interleukin-2 (IL-2) in preventing and treating ASD.

First the inventors evidenced the interest of IL-2 in a preventive setting. In a mouse model of MIA-induced autism, the inventors stimulated maternal Tregs with low dose IL-2 and evaluated the impact of this preventive treatment on offspring behaviors. They showed that maternal IL-2 treatment during pregnancy prevented abnormalities in early communication, repetitive behavior and social deficit and decreased isolated behaviors and increased all types of social approach and contact.

Second, the inventors then showed that administration of low dose IL-2 in the offspring of mouse model of MIA-induced autism similarly reduced early symptoms of autistic behaviors.

In light of the above, the invention provides IL-2 for use in treating autism spectrum disorder (ASD) in a subject. In a preferred embodiment, the subject is a child that has been diagnosed with ASD, preferably wherein the child is between 1 and 8 years old.

In another aspect, the invention provides IL-2 for use in preventing autism spectrum disorder (ASD) in a subject, wherein the IL-2 is administered to the subject who is an infant or a child, preferably wherein the infant or child is at risk of ASD.

In still another aspect, the invention provides IL-2 for use in preventing autism spectrum disorder (ASD) in an infant or a child, wherein the IL-2 is administered to a pregnant mother. In a preferred embodiment, the pregnant mother is at risk of having a child with ASD, optionally wherein the pregnant mother is affected with an infection or an autoimmune disease or disorder and/or optionally wherein the pregnant mother already had a child who developed ASD, or wherein maternal anti-fetal brain antibodies have been detected in the pregnant mother.

In a particular embodiment, IL-2 is conjugated to a PEG moiety, an Fc fragment, or any other moiety that improves the half-life of IL-2 or its diffusion in the central nervous system.

### LEGENDS TO THE FIGURES

FIGS. 1A, 1B, 1C are graphs that show the effect of low dose IL-2 administered in pregnant MIA mouse mothers on preventing disruption of the early social communication abilities of the offspring. FIG. 1A shows the number of vocalizations in 5 minutes in offspring of control mothers (“PBS”, n=32), MIA ASD models (“polyIC”, n=50), offspring of normal mothers administered with IL2 only (“IL2”, n=17), and in MIA ASD models administered with IL2 (“IL2+PolyIC”, n=26). FIG. 1B shows the duration of the vocalizations in the same mice offspring. FIG. 1C shows the modulation of the vocalizations in the same mice offspring. IL2 was administered as aldesleukin, 50000 U/day between E7.5 and E11.5. ANOVA *<0.05, **<0.01. Data are reported as means+/−SEM.

FIG. 2 is a graph that shows the effect of low dose IL-2 administered in pregnant MIA mouse mothers on preventing repetitive behaviors of the offspring, in a marble burying test at Week 6 (W6). In the marble burying test, 20 marbles are arrayed on the surface of clean bedding. The number of marbles buried in a 20 min session is scored (see Malkova et al, 2012), in offspring of control mothers (“PBS”, n=25), MIA ASD models (“polyIC”, n=17), offspring of normal mothers administered with IL2 only (“IL2”, n=19), and in MIA ASD models administered with IL2 (“IL2+PolyIC”, n=19). IL2 was administered as aldesleukin, 50000 U/day between E7.5 and E11.5. ANOVA *<0.05, **<0.01. Data are reported as means+/−SEM.

FIG. 3 is a graph that shows the effect of low dose IL-2 administered in pregnant MIA mouse mothers on social interaction of the offspring, in a 3-chamber test at Week 7 (W7). See Nadler et al, 2004. The index of social approach preference is determined in offspring of control mothers (“PBS”, n=16), MIA ASD models (“polyIC”, n=21), offspring of normal mothers administered with IL2 only (“IL2”, n=9), and in MIA ASD models administered with IL2 (“IL2+PolyIC”, n=14). IL2 was administered as aldesleukin, 50000 U/day between E7.5 and E11.5. ANOVA *<0.05, **<0.01. Data are reported as means+/−SEM.

FIGS. 4A to 4F are graphs that show the effect of low dose IL-2 administered in pregnant MIA mouse mothers on isolated behaviors and different types of social approach and contact, at Week 8 (W8), using a Live Mouse Tracker developed by de Chaumont et al, 2019. Data are reported as normalized on control mothers who received PBS only, in MIA ASD models (“polyIC”, n=12), offspring of normal mothers administered with IL2 only (“IL2”, n=10), and in MIA ASD models administered with IL2 (“IL2+PolyIC”, n=12). IL2 was administered as aldesleukin, 50000 U/day between E7.5 and E11.5. ANOVA *<0.05, **<0.01. Data are reported as means+/−SEM.

FIG. 5 is a graph that shows the effect of AAV-IL-2 administered at Week 3 (W3) in the offspring of MIA mice models on preventing repetitive behaviors, in a marble burying test at Week 6 (W6). In the marble burying test, 20 marbles are arrayed on the surface of clean bedding. The number of marbles buried in a 20 min session is scored (see Malkova et al, 2012), in offspring of control mothers (“PBS-AAV(e)”, n=8), MIA ASD models (“polyIC-AAV(e)”, n=8), normal mice administered with PBS-AAV(IL2) (“IL2”, n=19), and in MIA ASD models administered with AAV(IL2) (“PolyIC-AAV(IL2)”, n=15). PolyIC or PBS was administered at E12.5. AAV-IL2 or AAV-(e) were administered at W3. ANOVA *<0.05, **<0.01. Data are reported as means+/−SEM.

### DETAILED DESCRIPTION OF THE INVENTION

### Autism Spectrum Disorder

As used herein, the term “autism spectrum disorder” (ASD) is understood to cover a family of neurodevelopmental disorders characterized by deficits in social communication and interaction and restricted, repetitive patterns of behavior, interests or activities, e.g., as described in “American Psychiatric Association; Diagnostic and Statistical Manual of Mental Disorders (DSM-5) Fifth edition” (DSM-5).

The term “ASD patient” or “ASD subject” refers to a patient or subject having received a formal diagnosis of ASD or suspected of having ASD, i.e., subjects presenting behavioral characteristics of ASD and displaying clinical signs of ASD but who have not yet received a formal validation of their diagnostic. The person skilled in the art is well aware of how a patient may be diagnosed with ASD, in particular idiopathic ASD. For example, the skilled person may follow the criteria set up in “American Psychiatric Association; Diagnostic and Statistical Manual of Mental Disorders (DSM-5) Fifth edition” to give a subject a diagnosis of ASD. Likewise, ASD patients may have been diagnosed according to standardized assessments tools including but not limited to DSM IV, ICD-9, ICD-10, DISCO, ADI-R, ADOS, m-CHAT. In other cases, patients may have a well-established DSM-IV diagnosis of autistic disorder, or pervasive developmental disorder not otherwise specified (PDD-NOS).

### Subjects

The term “subject” or “patient” herein refers to humans, including newborns, infants (who are typically between two months and 1 year old) and children (who are between 1, 2, 3, 4, 5, 6, 7, 8 and up to 12, 13, 14, 15, 16, 17, 18, 19 years old, preferably between 1 and 10 years, preferably between 1 and 8 years old, still preferably between 1 and 6 years old).

In a curative setting, the subject is preferably a child who has been diagnosed with ASD, preferably wherein the child is between 1 and 8 years old, still preferably between 2 and 6 years old, or between 1 and 6 years old.

In a preventive setting, the term “subject” may preferably refer to an infant or a child, preferably wherein the infant or child is at risk of ASD. Preferably the subject is an infant or a child of less than 4 years old, less than 3 years old, still preferably less than 2 years old.

Infants or children at risk of ASD include infants or children who have a sibling with ASD or infants or children who were born premature babies, typically who were born less than 37 weeks of pregnancy, e.g., between 32 and 36 weeks, or between 23 to 32 weeks. Infants and children at risk of ASD also include infants or children with a history of MIA, namely infants or children whose mother has been affected with an infection during pregnancy, or is affected with an autoimmune disease or inflammatory disorder, as described below. Infants and children at risk of ASD further include infants and children showing genetic mutations or genetic variants associated with an increased risk of ASD, such as mutations in the FMR1 gene.

In another embodiment, the term “subject” refers to a pregnant mother, especially a pregnant mother who is at risk of having a child with ASD. Such women predisposed to have a child with ASD include a mother with ASD herself, or with a family history of ASD or of autoimmune disease, or a mother who is or has been affected with an infection during pregnancy, or is affected with an autoimmune disease or inflammatory disorder and/or a pregnant mother who already had a child who developed ASD, or a pregnant mother who has developed maternal anti-fetal brain antibodies. Anti-fetal brain antibodies are also known as “brain reactive” antibodies, namely antibodies that recognize antigens of the central nervous system.

Typical autoimmune diseases or inflammatory disorders include, but are not limited to, systemic lupus erythematosus (SLE), rheumatic arthritis, psoriasis, and autoimmune thyroid disease (Chen et al, 2016).

In a particular embodiment, mothers at risk include pregnant women who have experienced an episode of fever during the first or second trimester of pregnancy (Antoun et al, 2021), and/or who have experienced at least 3 episodes of fever, and/or a fever that lasted more than 7 days during their pregnancy.

### Preventive Settings

The term “preventing” or “prevention” herein refers to prophylactic treatment, by reducing the risk of the onset or development of the disease, especially in a subject who is asymptomatic but has been determined as being “at risk”, or susceptible to develop ASD. The term “preventing” or “prevention” encompasses reducing the development of at least one symptom of the disease, e.g., a deficit in social communication and interaction and restricted, repetitive patterns of behavior, interests or activities.

It is herein provided a method for preventing autism spectrum disorder (ASD) that is susceptible to develop ASD, which method comprises administering the IL-2 to
i) a subject in need thereof, who is an infant or a child; and/or
ii) a pregnant mother, so as to prevent ASD in the child she bears.

In embodiment (i), IL-2 is advantageously administered to the subject as early as possible, preferably in the newborn, infant or child of less than 2 years old.

In embodiment (ii), IL-2 is advantageously administered during or starting the first trimester of pregnancy. A preferred course of administration is a weekly administration during at least one month, preferably at least two months, still preferably between two and three months. In another preferred embodiment, the preventive treatment may comprise a first course that is also designated as an induction course, and a second course, that is a maintenance course. In a particular embodiment, the treatment may comprise at least a first course wherein the IL2 is administered once per day during at least about 2 or 3 consecutive days, preferably during 3 to 7, still preferably during 4 to 5 consecutive days, preferably followed by a maintenance dose, e.g., after about six days or about 1 to about 4 weeks.

### Curative Settings

It is herein provided a method for treating ASD in a subject in need thereof, which method comprises administering IL2 to the subject, preferably at a low dose as defined below.

The term “treating” or “treatment” means any improvement in the disease. It includes alleviating at least one symptom, or reducing the severity of the disease. Symptoms include a deficit in social communication and interaction and restricted, repetitive patterns of behavior, interests or activities.

In a preferred embodiment, it is herein described a method of treating an ASD in a subject, comprising administering the composition once or twice a week, or even once or twice a month, preferably by subcutaneous route.

### IL-2

As used herein, Interleukin-2 (IL-2) encompasses mammal wild type Interleukin-2, and active analogs thereof. Preferably, IL-2 is a human IL-2, or aldesleukin, as defined below.

Active analogs of IL-2 have been disclosed in the literature. An “analogue” designates a polypeptide comprising the native polypeptide sequence with one or more amino acid substitutions, insertions, or deletions. Muteins and pseudopeptides are specific examples of analogues. The IL-2 moiety of active variants generally has at least 75%, preferably at least 80%, 85%, more preferably at least 90% or at least 95% amino acid sequence identity to the amino acid sequence of the reference IL-2 polypeptide, for instance mature wild type human IL-2.

As used herein, “wild type IL-2” means IL-2, whether native or recombinant, comprising the 133 normally occurring amino acid sequence of native human IL-2, whose amino acid sequence is described in Fujita, et. al., PNAS USA, 80, 7437-7441 (1983). SEQ ID NO: 2 (133 amino acids) is the human IL-2 sequence less the signal peptide, consisting of an additional 20 N-terminal amino acids. SEQ ID NO:1 (153 amino acids) is the human IL-2 sequence including the signal peptide.

As used herein, “IL-2 mutein” means a polypeptide in which specific amino acid substitutions to the human mature interleukin-2 protein have been made. All numbering of the amino acids is made with respect to human mature interleukin-2 protein of SEQ ID NO: 2, unless otherwise indicated.

In some embodiments, the cysteine at position 125 is replaced with a neutral amino acid such as serine (C125S), alanine (C125A), threonine (C125T) or valine (C125V).

For example, elimination of the O-glycosylation site results in a more homogenous product when active variant is expressed in mammalian cells such as CHO or HEK cells.

In certain embodiments active variant comprises an additional amino acid mutation which eliminates the O-glycosylation site of IL-2 at a position corresponding to residue 3 of human IL-2. In one embodiment said additional amino acid mutation which eliminates the O-glycosylation site of IL-2 at a position corresponding to residue 3 of human IL-2 is an amino acid substitution. Exemplary amino acid substitutions include T3A, T3G, T3Q, T3E, T3N, T3D, T3R, T3K, and T3P. In a specific embodiment, said additional amino acid mutation is the amino acid substitution T3A.

The active analogs as used herein are capable of selectively promoting T-reg cell proliferation, survival, activation and/or function. “Regulatory T cells” or “Tregs” are T lymphocytes having immunosuppressive activity, characterized as CD4+CD25+Foxp3+ cells. “Effector T cells” (or “Teffs”) designate conventional T lymphocytes other than Tregs (sometimes also referred to as Tconv in the literature), which express one or more T cell receptor (TCR) and perform effector functions (e.g., cytotoxic activity, cytokine secretion, anti-self recognition, etc).

By “selectively promote,” it is meant that the active analog promotes the activity in T-reg cells but has limited or lacks the ability to promote the activity in non-regulatory T cells. Further described herein are assays to screen for active analogs that selectively promote T-reg cell proliferation, survival, activation and/or function. Methods for determining whether an IL-2 analog is active are available in the art. See e.g. WO2016/014428. An active analog is defined as an analog that shows an ability to stimulate Tregs, including analogs with an improved ability, or a similar ability, or even a reduced ability to stimulate Tregs when compared to wild-type IL-2 or aldesleukin (as defined below), to the extent it does not stimulate Teffs more than it stimulates Tregs. Methods for testing whether a candidate molecule stimulate T cells, Tregs in particular, or NK cells are well-known. Analogs may be tested for their ability to stimulate effector T cells (such as CD8+ T cells), CD4+Foxp3+ Tregs, or NK cells. In a preferred embodiment, the active analog shows a reduced ability to stimulate NK cells, compared to wild type IL2 or aldesleukin. Monitoring STAT5 phosphorylation is a simple way of assessing variants for their ability to preferentially stimulate Tregs over Teff, as described in Yu et al, Diabetes 2015; 64:2172-2183. In a particular embodiment, an analog is particularly useful when a given level of STAT5 phosphorylation is achieved with doses at least 10 times inferior for Tregs than for other immune cells, including Teffs.

Said active analogs induce signaling events that preferentially induce survival, proliferation, activation and/or function of Treg cells. In certain embodiments, the IL-2 analog retains the capacity to stimulate, in Treg cells, STAT5 phosphorylation and/or phosphorylation of one or more of signaling molecules downstream of the IL-2R, e.g., p38, ERK, SYK and LCK. In other embodiments, the IL-2 analog retains the capacity to stimulate, in Treg cells, transcription or protein expression of genes or proteins, such as FOXP3, Bcl-2, CD25 or IL-10, that are important for Treg cell survival, proliferation, activation and/or function. In other embodiments, the IL-2 analog exhibits a reduced capacity to stimulate endocytosis of IL-2/IL-2R complexes on the surface of CD25+ T cells. In other embodiments, the IL-2 analog demonstrates inefficient, reduced, or absence of stimulation of PI3-kinase signaling, such as inefficient, reduced or absent phosphorylation of AKT and/or mTOR (mammalian target of rapamycin). In yet other embodiments, the IL-2 analog retains the ability of wild type IL-2 to stimulate STAT5 phosphorylation and/or phosphorylation of one or more of signaling molecules downstream of the IL-2R in Treg cells, yet demonstrates inefficient, reduced, or absent phosphorylation of STAT5, AKT and/or mTOR or other signaling molecules downstream of the IL-2R in FOXP3−CD4+ or CD8+ T cells or NK cells. In other embodiments, the IL-2 analog is inefficient or incapable of stimulating survival, growth, activation and/or function of FOXP3−CD4+ or CD8+ T cells or NK cells.

In all cases, these analogs have the capacity to stimulate cell lines such as CTLL-2 or HT-2 which can be universally used to determine their biological activity. For instance, the biological activity of IL-2 may be determined by a cell-based assay performed on HT-2 cell line (clone A5E, ATCC® CRL-1841™) whose growth is dependent on IL-2. Cell growth in the presence of a range of test interleukin-2 product is compared with the growth recorded with IL-2 international standard (WHO 2nd International Standard for INTERLEUKIN 2 (Human, rDNA derived) NIBSC code: 86/500). Cell growth is measured after addition and transformation of [3-(4,5-dimethylthiazol-2-yl)-5-(3-carboxymethoxyphenyl)-2-(4-sulfophenyl)-2H-tetrazolium (inner salt, MTS) into formazan by active viable cells. Formazan concentration is then measured by spectrophotometry at 490 nm.

Examples of IL-2 analogs are disclosed, for instance, in EP109748, EP136489, U.S. Pat. No. 4,752,585; EP200280, EP 118617, WO99/60128, EP2288372, U.S. Pat. Nos. 9,616,105, 9,580,486, WO2010/085495, WO2016/164937.

For instance, certain mutations may result in a reduced affinity for the signaling chains of the IL-2 receptor (IL-2Rp/CD122 and/or IL-2Rγ/CD132) and/or a reduced capacity to induce a signaling event from one or both subunits of the IL-2 receptor. Other mutations may confer higher affinity for CD25 (IL-2Ra). In both cases, those mutations define active variants that preferentially induce survival, proliferation, activation and/or function of Treg. This property may be monitored using surface plasmon resonance.

Particular examples of useful analogs include IL-2 muteins which show at least one amino acid substitution at position D20, N30, Y31, K35, V69, Q74, N88, V91, or Q126, numbered in accordance with wild type IL-2, meaning that the chosen amino acid is identified with reference to the position at which that amino acid normally occurs in the mature sequence of wild type IL-2 of SEQ ID NO:2.

Preferred IL-2 muteins comprise at least one substitution at position D20H, D20I, D20Y, N30S, Y31H, K35R, V69AP, Q74, N88R, N88D, N88G, N88I, V91K, or Q126L.

In some embodiments, the IL-2 mutein molecule comprises a V91K substitution. In some embodiments, the IL-2 mutein molecule comprises a N88D substitution. In some embodiments, the IL-2 mutein molecule comprises a N88R substitution. In some embodiments, the IL-2 mutein molecule comprises a substitution of H16E, D84K, V91N, N88D, V91K, or V91R, any combinations thereof. In some embodiments, these IL-2 mutein molecules also comprise a substitution at position 125 as described herein. In some embodiments, the IL-2 mutein molecule comprises one or more substitutions selected from the group consisting of: T3N, T3A, L12G, L12K, L12Q, L 12S, Q13G, E15A, E15G, E15S, H16A, H16D, H16G, H16K, H16M, H16N, H16R, H16S, H16T, H16V, H16Y, L19A, L19D, L19E, L19G, L19N, L19R, L19S, L19T, L19V, D20A, D20E, D20H, D20I, D20Y, D20F, D20G, D20T, D20W, M23R, R81A, R81G, R81 S, R81T, D84A, D84E, D84G, D84I, D84M, D84Q D84R, D84S, D84T, S87R, N88A, N88D, N88E, N88I, N88F, N88G, N88M, N88R, N88S, N88V, N88W, V91D, V91E, V91G, V91 S, I92K, I92R, E95G, and Q126. In some embodiments, the amino acid sequence of the IL-2 mutein molecule differs from the amino acid sequence set forth in mature IL-2 sequence with a C125A or C125S substitution and with one substitution selected from T3N, T3A, L12G, L12K, L12Q L12S, Q13G, E15A, E15G, E15S, H16A, H16D, H16G, H16K, H16M, H16N, H16R, H16S, H16T, H16V, H16Y, L19A, L19D, L19E, L19G, L19N, L19R, L19S, L19T, L19V, D20A, D20E, D20F, D20G, D20T, D20W, M23R, R81A, R81G, R81 S, R81T, D84A, D84E, D84G, D84I, D84M, D84Q, D84R, D84S, D84T, S87R, N88A, N88D, N88E, N88F, N88I, N88G, N88M, N88R, N88S, N88V, N88W, V91D, V91E, V91G, V91 S, I92K, I92R, E95G, Q126I, Q126L, and Q126F. In some embodiments, the IL-2 mutein molecule differs from the amino acid sequence set forth in mature IL-2 sequence with a C125A or C125S substitution and with one substitution selected from D20H, D20I, D20Y, D20E, D20G, D20W, D84A, D84S, H16D, H16G, H16K, H16R, H16T, H16V, I92K, I92R, L12K, L19D, L19N, L19T, N88D, N88R, N88S, V91D, V91G, V91K, and V91S. In some embodiments, the IL-2 mutein comprises N88R and/or D20H mutations.

These substitutions can be used alone or in combination with one another. In some embodiments, the mutein comprises each of these substitutions. In some embodiments, the mutein comprises 1, 2, 3, 4, 5, 6, 7, or 8 of these mutations.

In some embodiments, the IL-2 mutein comprises a N88R or a N88D mutation, preferably N88R. In some embodiments, the IL-2 mutein comprises a C125A or C125S mutation. These substitutions can be used alone or in combination with one another. In some embodiments, the mutein comprises 1, 2, 3, 4, 5, 6, 7, or 8 of these mutations. In some embodiments, the mutein comprises each of these substitutions.

In a particular embodiment, the IL-2 moiety is aldesleukin. Aldesleukin is the active ingredient of Proleukin®. Aldesleukin is a variant of mature human IL-2 comprising two amino acid modifications as compared to the sequence of mature human IL-2 (SEQ ID NO:2): the deletion of the first amino acid (alanine) and the substitution of cysteine at position 125 by serine.

Conservative modifications and substitutions at other positions of IL-2 (i. e., those that have a minimal effect on the secondary or tertiary structure of the mutein) are encompassed. Such conservative substitutions include those described by Dayhoff in The Atlas of Protein Sequence and Structure 5 (1978), and by Argos in EMBO J., 8: 779-785 (1989). For example, amino acids belonging to one of the following groups represent conservative changes: -ala, pro, gly, gln, asn, ser, thr; -cys, ser, tyr, thr; -val, ile, leu, met, ala, phe; -lys, arg, his; -phe, tyr, trp, his; and -asp, glu.

Variants with mutations which disrupt the binding to the a subunit of IL-2R are not preferred, as those mutants may have a reduced capacity to stimulate Tregs.

In a particular aspect, the IL-2 molecule (including any active analog thereof) may be glycosylated, phosphorylated, fused to another polypeptide or molecule, polymerized, etc., or through chemical or enzymatic modification or addition to improve the properties of IL-2 (e.g., stability, specificity, etc.).

In a particular embodiment, the IL-2 is conjugated to a water-soluble polymer, such as polyethylene glycol (PEG). A preferred conjugate is described in patent application WO2012/065086, wherein the conjugate comprises a water-soluble polymer such as PEG covalently attached via a releasable linkage to an amine group of an IL-2 moiety.

In a particular embodiment, the IL-2 may be mutated at position D109C (with the C residue being capable of binding a PEG moiety), as described e.g. in international patent application WO2016/0025385.

In another particular embodiment, the IL-2 is fused to an immunoglobulin, preferably an IgG, preferably a human IgG, or preferably to a Fc region of an immunoglobulin. On may also use a particular fusion construct, that comprises two IL-2 proteins fused to one immunoglobulin, is disclosed e.g. in WO2014/023752 and WO2015/118016.

In another embodiment, the IL-2 is fused at the N-terminal end of a Fc moiety, either directly or preferably through a peptide linker, e.g. an 8 to 12 amino acid linker, as described e.g. in international patent application WO2016/014428.

In a particular embodiment, IL-2 is conjugated to a beta chain of the C4b-binding protein (C4BP) or at least one fragment or functional variant thereof that is capable of forming a dimeric protein, as described in international patent application WO2021/116444.

In a preferred embodiment, the IL2 moiety is fused to a fragment of the human C4BP β chain that comprises or consists of at least amino acids 194 to 252 or a longer fragment of C4BP that extends at the N-term up to at most amino acid 135. Preferably the IL2 moiety is fused at the N-terminus of C4BP β or said fragment thereof.

In preferred embodiments, functional variants of C4BP may comprise
a) a modified sequence of the fragment of C4BP, wherein less than 25 percent of the amino acids of the fragment, preferably less than 10 percent, have been cut out or replaced, in which the cysteines located in positions 202 and 216 as well as at least 3 amino acids upstream and downstream of each cysteine have been conserved; or
b) a modified sequence of the fragment of the C4BP, wherein a cysteine responsible for dimerization is substituted with an amino acid, preferably selected from alanine, valine, phenylalanine, proline, methionine, isoleucine, leucine and tryptophan, and another amino acid of the fragment is substituted with a cysteine;
or c) a sequence of the fragment of C4BP modified by insertion of a sequence which is heterologous to the beta chain, between the cysteines responsible for dimerization; or d) a sequence of the fragment of C4BP modified by cutting out amino acids between the cysteines responsible for dimerization.

In a preferred embodiment, it is provided a fusion protein wherein one IL2 moiety is fused at the N-term of C4BP β or of said fragment thereof, and another IL2 moiety is fused at the C-term of C4BP β or of said fragment thereof. According to such embodiment, the fusion protein comprises the following sequence from N- to C-term: IL2—C4BP IL2. The IL2 moiety and C4BP β or said fragment thereof may be fused in frame (directly) or through an amino acid linker, preferably a polyG linker.

The IL-2 may be used alone or in combination with any other therapeutically active agent.

### Production Methods

The IL2 can be typically produced by DNA recombinant technique in a suitable expression vector or by a RNA molecule.

The expression vector is selected as a function of the host cell into which the construct is introduced. Preferably, the expression vector is selected from vectors that allow expression in eukaryotic cells, especially from chromosomal vectors or episomal vectors or virus derivatives, in particular vectors derived from plasmids, yeast chromosomes, or from viruses such as baculovirus, papovirus or SV40, retroviruses, Adenoviruses, Adeno-associated Viruses, or combinations thereof, in particular phagemids and cosmids. In a particular embodiment, it is a vector allowing the expression of baculovirus, capable of infecting insect cells.

If necessary, the sequence coding for the fusion polypeptide also comprises, preferably in its 5′ portion, a sequence coding for a signal peptide for the secretion of fusion polypeptide. Conventionally, the sequence of a signal peptide is a sequence of 15 to 20 amino acids, rich in hydrophobic amino acids (Phe, Leu, Ile, Met and Val).

The vector comprises all of the sequences necessary for the expression of the sequence coding for the fusion polypeptide. In particular, it comprises a suitable promoter, selected as a function of the host cell into which the construct is to be introduced.

Within the context of the invention, the term “host cell” means a cell capable of expressing a gene carried by a nucleic acid which is heterologous to the cell and which has been introduced into the genome of that cell by a transfection method.

Preferably, a host cell is a eukaryotic cell. A eukaryotic host cell is in particular selected from yeast cells such as S cerevisiae, filamentous fungus cells such as Aspergillus sp, insect cells such as the S2 cells of Drosophila or sf9 of Spodoptera, mammalian cells and plant cells. Mammalian cells which may in particular be cited are mammalian cell lines such as CHO, COS, HeLa, C127, 3T3, HepG2 or L(TK−) cells. In a preferred implementation, said host cells are selected from eukaryotic cell lines, preferably Sf9 insect cells. Methods for preparing recombinant dimeric proteins in sf9 insect cells are described in U.S. Pat. No. 7,884,190. Any transfection method known to the skilled person for the production of cells expressing a heterologous nucleic acid may be used to carry out step a) of the method. Transfection methods are, for example, described in Sambrook et al, 2001, “Molecular Cloning: A Laboratory Manual”, 3rd edition, Cold Spring Harbor Laboratory Press, Cold Spring Harbor, N.Y.

Alternatively, the IL2 can be produced by chemical peptide synthesis. For instance, the protein can be produced by the parallel synthesis of shorter peptides that are subsequently assembled to yield the complete sequence of the protein with the correct disulfide bridge. A synthesis of IL-2 is illustrated for instance in Asahina et al., Angewandte Chemie International Edition, 2015, Vol. 54, Issue 28, 8226-8230.

### In Vivo Expression

In another embodiment, the IL2 may be expressed in vivo, after administering the subject with a nucleic acid encoding said chimeric protein.

In a preferred embodiment, the nucleic acid is carried by an RNA or a viral vector, such as an adeno-virus associated virus (AAV), e.g. AAV8 or AAV9 (see Wang et al, 2019).

Typically, a recombinant AAV vector comprises an AAV capsid and an expression cassette comprising a promoter and a nucleic acid that encodes the desired transgene, herein IL2.

The expression may be constitutive or inducible. The promoter may have an ubiquitous expression or, preferably, a cell specific expression, e.g. it may be a GFAP promoter for expression in brain cells (astrocytes, see Yshii et al, 2022).

The quantity of recombinant vector that is administered is adjusted by the skilled person so that the IL2 transgene is expressed at a blood level that allows an increase of the Treg/Teff ratio, or a stimulation of Tregs without substantial stimulation of Teffs. Typically 10¹⁰ viral genomes may be administered.

### Dosage and Regimen

The dosage is chosen so as to effectively expand and activate Tregs without substantially activating Teffs.

This is of particular pertinence when the IL2 is wild-type IL2 or aldesleukin or a mutein that retains some ability to activate Teffs if used at a high dosage.

Generally speaking, doses that allow a 1.2, 1.5, 2, 3, 4 or 5-fold increase of the number of Tregs are preferred. The standard measure of an amount IL-2 is the International Unit (IU), which technically is not a fixed weight but the amount that produces a fixed biological effect in a specific cell proliferation assay, as determined by the World Health Organization (WHO). The reason is that i) the weight varies depending on the exact sequence of the molecule and its glycosylation profile, and ii) what matters is the activity, not the weight of the molecule.

The principle of the International Unit is precisely to provide a standard to which any IL-2 molecule can be compared (regardless of their source, or their sequence, including wild-type or active variant sequences).

In practice, the WHO provide ampoules containing an IL-2 molecule that has been calibrated and serves as the reference to determine the dosage of a given preparation of IL-2 (again regardless of the source or sequence of said IL-2) defined by its potency. For instance, to determine the dosage of a given preparation of IL-2, the biological activity of the candidate IL-2 preparation is measured in a standard cell proliferation assay using an IL-2 dependent cell line, such as CTLL-2, and compared with the biological activity of the standard. The cells are grown in the presence of different doses of the standard. A dose-response effect of IL-2 is established, where the dose of IL-2 is plotted on the X axis as IU and the measure of proliferation (pr) is on the Y axis. When one wants to determine the activity of any IL-2 product of unknown activity, the product is used to grow the IL-2 dependent cells and the proliferation is measured. The pr value is then plotted on the Y axis and from that value a line parallel to the X axis is drawn. From the point of intersection of this line with the dose response line, a line parallel to the Y axis is then drawn. Its intersection with the X axis provides the activity of the candidate IL-2 product in IU.

Any change of the WHO standard ampoules does not impact the International Unit nor the determination of a dosage of any IL-2 preparation.

The 1st standard (WHO international Standard coded 86/504, dated 1987) contained a purified glycosylated IL-2 derived from Jurkat cells and was arbitrarily assigned a potency of 100 IU/ampoule. As the stocks of the 1st international standard (IS) were running low, the WHO had to replace it. The WHO provided another calibrated IL-2 ampoule, this time produced using E. coli. The 2nd standard ampoules contained 210 IU of biological activity per ampoule. The change of standard ampoules does not mean that the IU changes. So, determining the dosage of a test IL-2 preparation will not vary whether one uses the 1st standard ampoule or the 2nd standard ampoule, or a subsequent standard ampoule, as a reference.

In a preferred embodiment, the treatment may comprise a first course that is also designated as an induction course, and a second course, that is maintenance course.

In a particular embodiment, the treatment may comprise at least a first course wherein the pharmaceutical composition is administered once per day during at least about 2 or 3 consecutive days, preferably during 3 to 7, still preferably during 4 to 5 consecutive days, preferably followed by a maintenance dose, e.g. after about six days or about 1 to about 4 weeks.

The maintenance dose may be typically administered during at least one month, preferably at least about 3 months, still preferably at least about 6 months. In a preferred embodiment, the maintenance dose is administered between about 3 months and about 12 months, preferably between about 6 months and about 12 months.

In a preferred embodiment, the maintenance treatment consists of an administration of the pharmaceutical composition once or twice a week, or every one or two weeks, or once a month.

In a preferred embodiment, the maintenance treatment consists of an administration of interleukin-2 once or twice a week, every one or two weeks, or once a month during a period of at least one month, preferably from about 3 months to about 12 months.

Preferably the maintenance dosage is substantially the same as the first course dosage, or it can be a lower or higher dosage.

In another embodiment, the IL-2 is given every other day for 1 to 2 weeks, in cycles that can be repeated after break of administration that can last from 3 days to 3 months, preferably from one to 4 weeks.

In a particular embodiment, the subject is administered with IL-2 as the single active ingredient effective in treating ASD or a symptom thereof.

Typically, the dosage according to the invention is at low dose, e.g. below 3.5 Million IU/day, more preferably below 3.0 Million IU/day, even more preferably below 2.5 Million IU/day, further preferably below 2.0 Million IU/day. This dosage effectively activates Tregs without substantially activating Teffs. The consequence is a dramatic increase in the Treg/Teff balance in the subject. At this dosage IL-2 substantially avoids side effects, while very substantially inducing Tregs.

According to the invention, IL-2 is preferably administered at a dosage ranging from about 0.1 to 3 MIU/day, preferably 0.5 to 2.5 MIU/day, still preferably 1 MIU/day to about 2 MIU/day.

In a preferred embodiment, particularly advantageous for subcutaneous administration, IL-2 is administered at a dose of 1, 1.5 or 2 MIU/day.

Exemplary dosages are between 0.1 to 3 MIU, preferably 0.1 to 1.5 MIU, still preferably 0.25 to 1 MUI. Preferred dosages are:
3.5 M IU/day,
3.0 M IU/day,
2.5 M IU/day,
2.0 M IU/day,
1.5 M IU/day,
1.0 M IU/day,
0.5 M IU/day,
0.3 MIU/day,
0.1 M IU/day,
0.05 M IU/day,
0.02 M IU/day, or
0.01 M IU/day.

These dosages may be combined, depending on the subject and evolution of the disease.

The effective dosage can be adjusted by the practitioner, based on the profile and age of the subject to whom the IL-2 is administered, especially when the subject is a newborn or an infant. Typically IL-2 is administered at a dose of about 0.05 to about 2 MIU/m²/day, preferably 0.2 to about 1 MIU/m²/day.

The amount of IL-2 to administer thus preferably depends on the body surface area of the subject. The body surface area (BSA) is the measured or calculated surface of a human body.

Various calculations have been published to arrive at the BSA without direct measurement:

The Dubois & Dubois formula (1916) is commonly used in adults:

$$BSA ( {m}^{2} ) = 0.0 \; 07184 \times weight {( kg )}^{0.425} \times height {( cm )}^{0.725} = \frac{weight {( kg )}^{0.425} \times height {( cm )}^{0.725}}{139.2}$$

Another commonly used formula is the Mosteller formula (1987) adopted for use by the Pharmacy and Therapeutics Committee of the Cross Cancer Institute, Edmonton, Alberta, Canada:

$$BSA ( {m}^{2} ) = \sqrt{\frac{weight ( kg ) \times height ( cm )}{3600}} = \frac{weight {( kg )}^{0.5} \times height {( cm )}^{0.5}}{60}$$

It is more particularly used in children.

Average BSA is generally taken to be 1.73 m² for an adult.

Average BSA values

| | | |
|---|---|---|
| Neonate (Newborn) | 0.25 | m² |
| Child 2 years | 0.5 | m² |
| Child 9 years | 1.07 | m² |
| Child 10 years | 1.14 | m² |
| Child 12-13 years | 1.33 | m² |
| For men | 1.9 | m² |
| For women | 1.6 | m² |

According to the invention, the treatment—be it preventive or curative—typically comprises at least a first course wherein interleukin-2 is administered once per day during at least about 2 or 3 consecutive days, preferably during 3 to 7, still preferably during 4 to 5 consecutive days, preferably followed by a maintenance dose after about five or six days or about 1 to about 4 weeks.

The maintenance dose is typically administered during at least one month, preferably at least about 3 months, still preferably at least about 6 months. In a preferred embodiment, the maintenance dose is administered between about 3 months and about 12 months, preferably between about 6 months and about 12 months.

In a preferred embodiment, the maintenance treatment consists of an administration of interleukin-2 once or twice a week, or every one or two weeks.

In a preferred embodiment, the maintenance treatment consists of an administration of interleukin-2 once or twice a week, every one or two weeks, during a period of at least one month, preferably from about 3 months to about 12 months.

Preferably the maintenance dosage is substantially the same as the first course dosage, or it can be a lower or higher dosage.

In a preferred embodiment, the treatment comprises at least a first course wherein interleukin-2 is administered at a dosage of at most 3.5 MIU/day, preferably about 1 to about 2 MIU/day, preferably 1-1.5 MIU/day, once per day during 2 or 3 to 7 days, preferably 5 days, followed by a maintenance dose after one to two weeks, of about 1 to about 2 MIU/day, preferably 1-1.5 MIU/day every 2 weeks, during at least three months, preferably at least six months.

In another embodiment, especially when the IL-2 molecule is a variant with longer half-life and/or is conjugated to a moiety that improves the half-life of the conjugate, the regimen is adjusted to the extent the Treg increase during maintenance remains at least 1.2, 1.5, 1.7, 1.9, or at least twice the baseline Treg level. For that purpose, in an embodiment, the regimen may be defined as a first (induction) course consisting of a dosage of at most 3.5 MIU one per day during 1 to 3 days, followed by a maintenance course after 1 to 4 weeks. Preferably the maintenance course then consists in an administration of IL-2 at a maintenance dosage, once every 2 weeks to once a month, during about 1 month, preferably 3 months, still preferably 6 months, or more.

The treatment is typically repeated, i.e., the above low doses IL-2 are administered several times to a subject, to progressively achieve the most substantial benefit. Treatment effect can be monitored by Treg measurements and dose and administration schedule adjusted accordingly.

Maintenance dosage can be administered from two to eight weeks after the initiating cycle is completed. Preferably the maintenance dose is the same as the initiating dose.

In a particular embodiment, especially when the subject is a newborn, an infant or a child, the method comprises administering at least a first course wherein a dose of no more than 0.2 MUI/m² of IL-2 is administered once a day during at least 3 consecutive days, preferably during 3 to 7, still preferably 4 to 5 consecutive days, followed by a maintenance dose of no more than 0.2 MUI/m² after one to three weeks, which maintenance dose can be repeated e.g. every one to three weeks.

### Administration Forms and Routes

Il-2 may be administered using any convenient route, including parenteral, e.g. intradermal, subcutaneous, or intranasal route. The subcutaneous route is preferred. Oral, sublingual or buccal administrations are also encompassed.

IL-2 is typically administered in association (e.g., in solution, suspension, or admixture) with a pharmaceutically acceptable vehicle, carrier or excipient. Suitable excipients include any isotonic solution, saline solution, buffered solution, slow release formulation, etc. Liquid, lyophilized, or spray-dried compositions comprising IL-2 or analogues thereof are known in the art and may be prepared as aqueous or nonaqueous solutions or suspensions. Preferably the pharmaceutical compositions comprise appropriate stabilizing agents, buffering agents, bulking agents, or combinations.

An example of an IL-2 formulation suitable for a subcutaneous injection is described in international patent application WO2017/068031.

The Examples illustrate the invention without limiting its scope.

### Example 1: Preventive Setting

Mouse model of ASD

In the well-established rodent maternal immune activation model (Smith et al, 2007), offspring from pregnant mice injected intra-peritoneally with synthetic dsRNA [poly(I:C), also designated PolyIC], a mimic of viral infection, exhibit behavioral symptoms reminiscent of ASD: social deficits, abnormal communication and repetitive behaviors (Malkova et al, 2012).

### Protocol

Pregnant female mice were treated with either IL-2 (aldesleukin, 50,000 IU daily s.c.) from E7.5 to E11.5 or with PBS (control), before IP injection of Poly (I:C) at E12.5 (5 mg/kg).

The inventors then evaluated the reversibility of autistic symptoms by different techniques (i) ultra sound vocalization recording at D7 for measuring early social communication abnormalities (ii) marble burying test at W6 for measuring restricted and stereotyped behaviors (iii) 3-chamber test at W7 for measuring appetence in social interactions.

Finally, at W8, the inventors used the live mouse tracker (LMT), as described in de Chaumont et al, 2019, which allowed to monitor various parameters of social interaction, to evaluate long-term behavior (>14 h of free interactions) using a machine learning algorithm. The protocol provides a real-time analysis of the behavior of mice housed in groups of up to four over several days and in enriched environments. The method combines computer vision through a depth-sensing infrared camera, machine learning for animal and posture identification, and radio-frequency identification to monitor the quality of mouse tracking. This system detected up to 4 animals (2 controls and 2 of the experimental condition to be tested) in the same field of observation. All the raw behavioral data was then analyzed using Python software (lmt-analysis package), to provide a phenotypic profile for each animal.

### Results

The inventors observed that preventive treatment with ld-IL2 during pregnancy increased maternal Tregs and prevented maternal IL-17 secretion related to Poly (I:C) injection (data not shown).

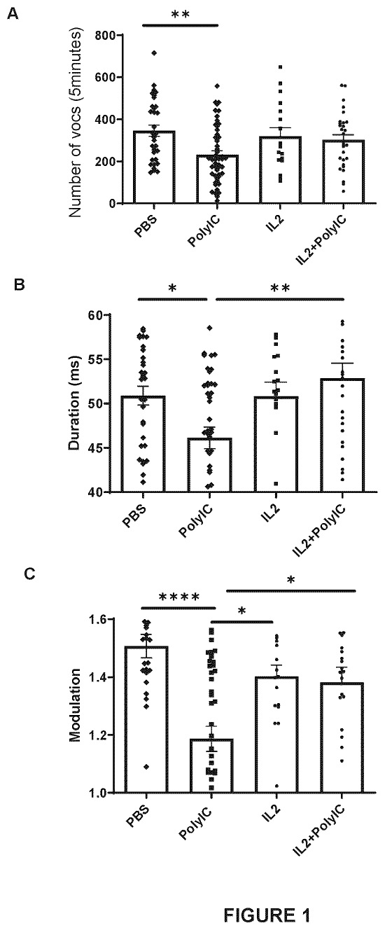

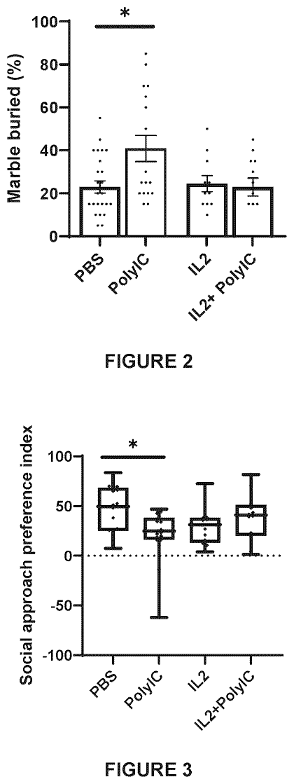

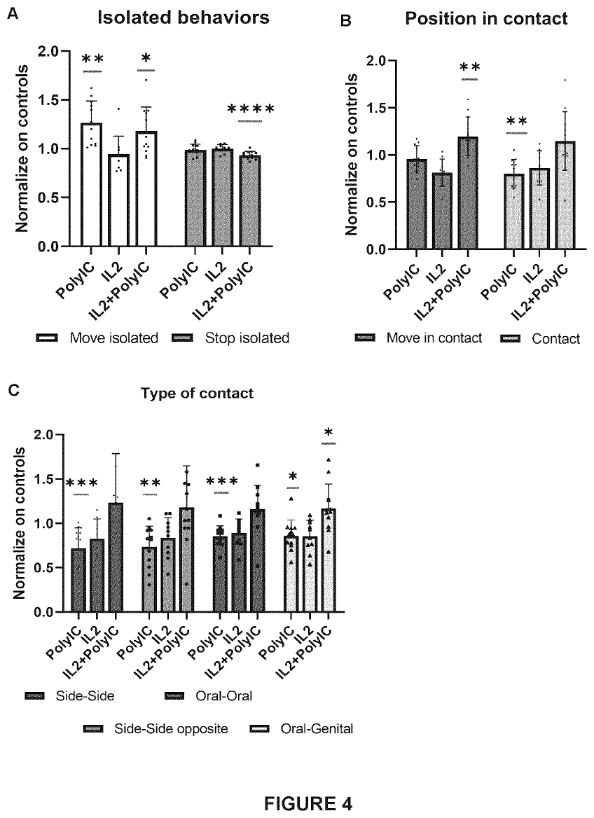

The inventors then observed that preventive treatment with ld-IL2 during pregnancy prevented both quantitative and qualitative abnormalities in early communication. See FIGS. 1A to 1C. Maternal Ld-IL2 also prevented repetitive behavior (FIG. 2) and social deficit in the offspring (FIG. 3).

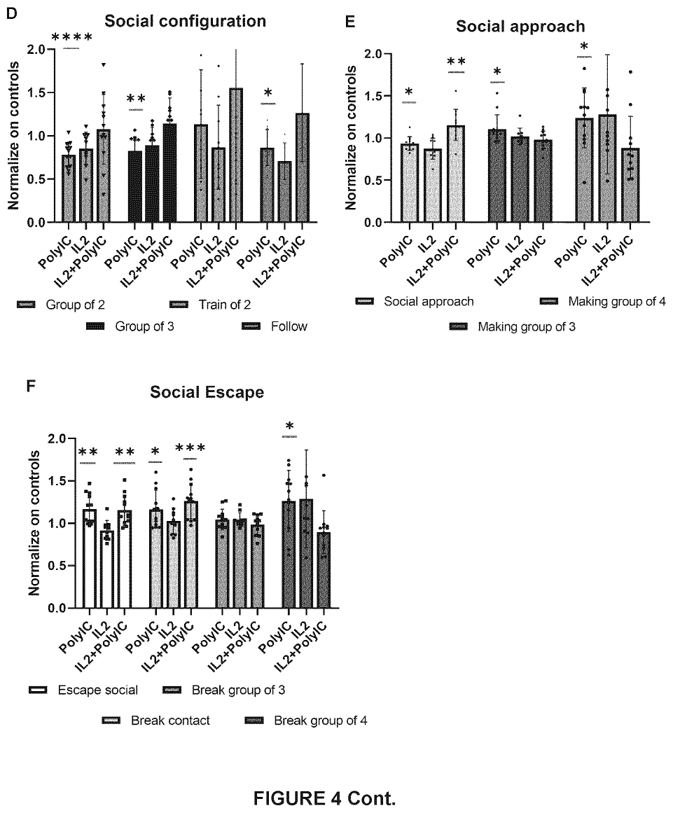

On the LMT, ld-IL2, decreased isolated behaviors and increased all types of social approach and contact (see FIGS. 4A to 4F).

The inventors further observed that preventive treatment with ld-IL2 during pregnancy reversed the reduction of Tregs in the offspring (data not shown).

Altogether these results show that, in a mouse model of MIA-induced autism, maternal Treg stimulation with low dose IL-2 prevents ASD onset in the offspring.

### Example 2: Curative Setting

Recombinant adeno-associated virus generation and in vivo administration Recombinant rAAV8 vectors were generated by triple transfection of human embryonic kidney 293 T cells, as described previously (Churlaud et al., 2014). Transgenes used were luciferase (LUC) and murine IL-2 (IL-2) driven by the hybrid cytomegalovirus enhancer/chicken beta-actin constitutive promoter (CAG). Mice were injected once intraperitoneally with 1×10¹⁰ viral genomes (vg) of rAAVs (AAV8-CAG-IL2, herein designated AAV(IL2) or AAV8-CAG-LUC, herein designated AAV(e)) diluted in 100 μl of 0.1 M phosphate-buffered saline (PBS).

### Protocol

Pregnant female mice were injected with either synthetic dsRNA [poly(I:C), also designated PolyIC, at 5 mg/kg] or PBS (control) at E12.5.

Using ultra sound vocalization recording for measuring early social communication abnormalities at D7, the inventors then checked that the offspring of the PolyIC mice showed autistic symptoms.

At W3, the offspring then received an administration of AAV-IL2 or AAV-(e), before running a marble burying test at W6 for measuring restricted and stereotyped behaviors.

At W5, the inventors further observed an increase of Tregs in offspring treated with AAV-IL2 (data not shown).

### Results

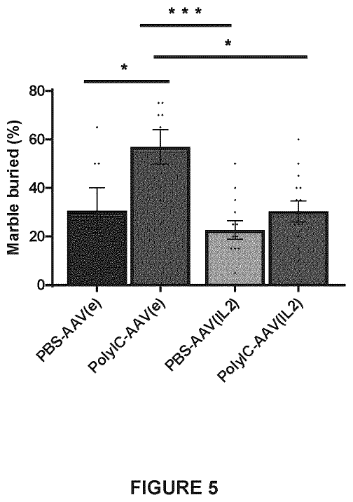

The inventors observed that curative treatment with AAV(IL2) prevented repetitive behavior in the ASD offspring (FIG. 5).

### REFERENCES

Antoun et al. Fever during pregnancy as a risk factor for neurodevelopmental disorders: results from a systematic review and meta-analysis Mol Autism. 2021, 12(1):60. doi: 10.1186/s13229-021-00464-4.
Chen et al, Maternal autoimmune diseases and the risk of autism spectrum disorders in offspring: A systematic review and meta-analysis, Behavioural Brain Research, 2016, 296:61-69.
Churlaud et al. Sustained stimulation and expansion of Tregs by IL2 control autoimmunity without impairing immune responses to infection, vaccination and cancer. Clin Immunol 2014; 151:114-26.
De Chaumont et al., Real-Time Analysis of the Behaviour of Groups of Mice via a Depth-Sensing Camera and Machine Learning. Nature Biomedical Engineering 2019, 3 (11): 930-42.
Dubois & Dubois Arch Intern Med 1916, 17:863
Estes & McAllister, Immune mediators in the brain and peripheral tissues in autism spectrum disorder, Nat Rev Neurosci, 2015, 16(8):469-86.
Estes & McAllister. Maternal immune activation: Implications for neuropsychiatric disorders Science, 2016, 353(6301):772-7.
Malkova et al. Maternal immune activation yields offspring displaying mouse versions of the three core symptoms of autism. Brain Behav. Immun. 2012, 26:607-616.
Mosteller R D. Simplified calculation of body-surface area. N Engl J Med 1987; 317:1098
Nadler et al. Automated apparatus for quantitation of social approach behaviors in mice. Genes Brain Behav. 2004; 3:303-314.
Smith et al, Maternal immune activation alters fetal brain development through interleukin-6. J Neurosci. 2007, 27:10695-10702.
Wang et al. Adeno-associated virus vector as a platform for gene therapy delivery. Nat Rev Drug Discov 2019, 18: 358-378.
Yshii, et al. Astrocyte-targeted gene delivery of interleukin 2 specifically increases brain-resident regulatory T cell numbers and protects against pathological neuroinflammation. Nat Immunol 2022, 23: 878-891.

1. Interleukin-2 (IL-2) for use in treating autism spectrum disorder (ASD) in a subject.

2. The IL-2 for use according to claim 1, wherein the subject is a child who has been diagnosed with ASD, preferably wherein the child is between 1 and 8 years old.

3. Interleukin-2 (IL-2) for use in preventing autism spectrum disorder (ASD) in a subject, wherein the IL-2 is administered to the subject who is an infant or a child, preferably wherein the infant or child is at risk of ASD.

4. Interleukin-2 (IL-2) for use in preventing autism spectrum disorder (ASD) in an infant or a child, wherein the IL-2 is administered to a pregnant mother.

5. The IL-2 for use according to claim 4, wherein the pregnant mother is at risk of having a child with ASD, optionally wherein the pregnant mother is with ASD herself, or is or has been affected with an infection during pregnancy, or is affected with an autoimmune disease or inflammatory disorder and/or optionally wherein the pregnant mother already had a child who developed ASD, or with anti-fetal brain antibodies.

6. The IL-2 for use according to claim 4 or 5, wherein the IL-2 is administered during the first trimester of pregnancy.

7. The IL-2 for use according to any of claims 1 to 6, wherein the IL-2 is administered at a maximum dose of 3.5 MIU/day or less, preferably 3 MIU/day or less, still preferably 2 MIU/day or less, still preferably at a dose of between 0.1 and 1.5 or 2 MUI/day.

8. The IL-2 for use according to any of claims 1-3, wherein the IL-2 is administered to a child at a dose of between 0.1 and 1 MUI/day, or between 0.5 and 1 MIU/day.

9. The IL-2 for use according to any of claims 1 to 8, wherein the IL-2 is administered repeatedly, preferably in a first course that preferably consists of at least one daily administration during 1 to 7 days, followed by a maintenance course after at least 5 days, which maintenance course preferably comprises or consists of at least one administration once every week during 1 week to 3 months.

10. The IL-2 for use according to any of claims 1 to 9, wherein said IL-2 is human IL-2 or aldesleukin or an active analogue thereof, wherein said active analogue has at least 85% or 90% amino acid identity with human wild-type IL-2, and is capable of activating Tregs, still preferably wherein said analogue is preferably an IL2 mutein that comprises a substitution at position N88 of SEQ ID NO: 2, still preferably substitution N88R or N88D.

11. The IL-2 for use according to any of claims 1 to 10, wherein said IL-2 is conjugated to a PEG moiety, an Fc fragment, or any other moiety that improves the half-life of IL-2 or its diffusion in the central nervous system.

12. The IL-2 for use according to claim 11, wherein said IL-2 is conjugated to a beta chain of the C4b-binding protein (C4BP ) or at least one fragment or functional variant thereof that is capable of forming a dimeric protein, preferably wherein the fragment of C4BP comprises, or consists of, amino acid residues 194 to 252 of C4BP or a longer fragment of C4BP that extends at the N-term up to at most amino acid 135.

13. The IL-2 for use according to any of claims 1 to 12, wherein said IL-2 is expressed in vivo after administration with a nucleic acid encoding said IL-2.

14. The IL-2 for use according to claim 13, wherein the nucleic acid is carried by an RNA or a viral vector, preferably an adeno-virus associated virus (AAV).

15. The IL-2 for use according to any of claims 1 to 14, wherein said IL-2 is administered by subcutaneous, intramuscular or intradermal route.


SEQUENCE LISTING (2 sequences)

SEQ ID NO: 1 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 2 - protein, 133 aa, Homo sapiens [sequence omitted]